(19) United States
(12) Patent Application Publication
(10) Pub. No.: US 2025/0269360 A1
(43) Pub. Date: Aug. 28, 2025

(54) CATALYTIC AEROBIC OXIDATIONS

(71) Applicant: Robert William McGaff, LaCrosse, WI (US)

(72) Inventor: Robert William McGaff, LaCrosse, WI (US)

(21) Appl. No.: 19/060,720

(22) Filed: Feb. 23, 2025

Related U.S. Application Data
(60) Provisional application No. 63/557,247, filed on Feb. 23, 2024.

(51) Int. Cl. B01J 31/18

(52) U.S. Cl. CPC B01J 31/183; B01J 2231/72; B01J 2231/763; B01J 2531/842

(57) ABSTRACT

The present disclosure provides a method of oxidizing organic substrates using molecular oxygen under pressure. The method involves contacting an organic substrate with an iron (III)-based phthalocyanine catalyst. Molecular oxygen is utilized as an oxidant and is provided at a pressure greater than atmospheric pressure to provide an oxidized product.

### CROSS-REFERENCE TO RELATED APPLICATION

This application claims the benefit of priority to U.S. Provisional Patent Application Ser. No. 63/557,247, filed Feb. 23, 2024, the disclosure of which is incorporated herein in its entirety by reference.

### BACKGROUND

Commodity chemicals, also known as bulk chemicals or basic chemicals, are essential raw materials used in a wide variety of industries. These materials form the backbone of the chemical industry. Commodity chemicals are typically manufactured at large scale and high production volumes in order to access low per unit prices. Efficient process chemistry can be particularly important for achieving low and competitive prices. Fine chemicals, prepared from or during preparation of commodity chemicals, represent higher value compounds used in more specialized applications. For fine chemical production, production processes that are efficient and versatile can be extremely important for facilitating economical production.

Oxidation reactions are extremely common and useful for chemical production. Examples of useful oxidation reactions include conversion of alcohols to ketones. However, many oxidation reactions can suffer from requiring problematic oxidation catalysts, which can involve harmful heavy metals or expensive precious metals. Others can suffer from requiring environmentally damaging solvents or co-reactants.

There remains a need for improved oxidation reactions and new approaches to chemical production.

### SUMMARY OF THE INVENTION

The present disclosure provides a method of oxidizing an organic substrate, comprising contacting an organic substrate with an iron (III)-based bridged phthalocyanine catalyst in a pressure vessel and providing O₂ at a pressure greater than atmospheric pressure to provide an oxidized product.

Advantages, some of which are unexpected, are achieved by various aspects of the present disclosure. For example, the present disclosure describes various oxidation methods that utilize high pressure conditions with molecular oxygen as the oxidant. Molecular oxygen can be readily sourced from air, thus representing a freely available and environmentally desirable oxidant source. As another example, the present disclosure describes various oxidation methods that utilize iron-based catalysts, which utilize inexpensive, earth-abundant iron, rather than expensive metals.

In various aspects, the disclosure provides oxidation methods that surprisingly and advantageously achieve selectivity when using high pressure conditions and molecular oxygen as an oxidant. Additionally, the disclosure achieves a general process that is tunable for oxidation of a wide range of organic substrates. It is especially surprisingly that, in various aspects, the high-pressure oxidation method has the advantage of high turnover frequency, high turnover number, and/or high yield.

### BRIEF DESCRIPTION OF FIGURES

FIG. 1 shows an example structure of a bridged phthalocyanine complex catalyst, where the structure corresponds to the catalyst “FeLX” when L is a mixture of H₂O and MeOH, and corresponds to the catalyst “FeLX8” when L is H₂O only.

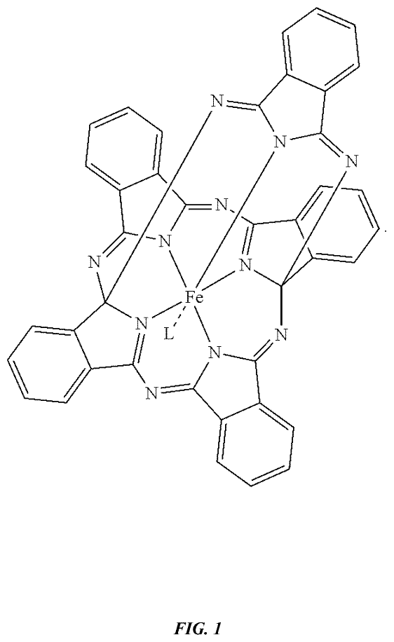

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges 0.1% to 0.5%, 1.1% to 2,2%, 3,3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. For example, an oxygen-containing group such as an alkoxy group, aryloxy group, aralkyloxy group, oxo (carbonyl) group, a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)_{0.2}N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH) N(R)₂, C(O)N(OR)R, C(═NOR)R, and substituted or unsubstituted (C₁-C₁₀₀) hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enmities; and other heteroatoms in various other groups. Non-limiting examples of substituents that can be bonded to a substituted carbon (or other) atom include F, Cl, Br, I, OR, OOR, OC(O)N(R)₂, CN, NO, NO₂, ONO₂, azido, CF₃, OCF₃, R, O(oxo), S(thiono), C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)_{0.2}N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH) N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 2 carbon atoms, 1 to 12 carbons or, in some aspects, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some aspects, from 2 to 8 carbon atoms. Examples include, but are not limited to vinyl, —CH═CH(CH₃), —CH═C(CH₃)₂, —C(CH₃)═CH₂, —C(CH₃)—CH(CH₃), —C(CH₂CH₃)—CH₂, cyclohexenyl, cyclopentenyl, cyclohexadienyl, butadienyl, pentadienyl, and hexadienyl, among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroatyl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acrylipyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some aspects, the cycloalkyl group can have 3 to about 8-12 ring members, whereas in other aspects the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl naphthacenyl, chrysenyl, biphenylenyl, anthracenyl, and naphthyl groups. In some aspects, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3—, 4-, 5-, or 6-positions of the phenyl ring, or a naphthyl group substituted at any one or more of 2- to 8-positions thereof.

The term “heterocyclyl” as used herein refers to aromatic and non-aromatic ring compounds containing three or more ring members, of which one or more is a heteroatom such as, but not limited to, N, O, and S.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “hydrocarbon” or “hydrocarbyl” as used herein refers to a molecule or functional group, respectively, that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b}) hydrocarbyl, wherein a and b are integers and mean having any of a to b number of carbon atoms. For example, C₁-C₄) hydrocarbyl means the hydrocarbyl group can be methyl(C₁), ethyl(C₂), propyl(C₃), or butyl(C₄), and (C₀-C_{b}) hydrocarbyl means in certain aspects there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “room temperature” as used herein refers to a temperature of about 15° C. to 28° C.

The term “standard temperature and pressure” as used herein refers to 20° C. and 101 kPa.

The structures disclosed herein, in all of their aspects are intended to include only “chemically feasible” structures, and any recited structures that are not chemically feasible, for example in a structure shown with variable atoms or groups, are not intended to be disclosed or claimed herein. By “chemically feasible” is meant a bonding arrangement or a compound where the generally understood rules of organic structure are not violated; for example, a structure within a definition of a claim that would contain in certain situations a pentavalent carbon atom that would not exist in nature would be understood to not be within the claim.

### Catalyst

The present disclosure relates to use of a catalyst according to Formula I.

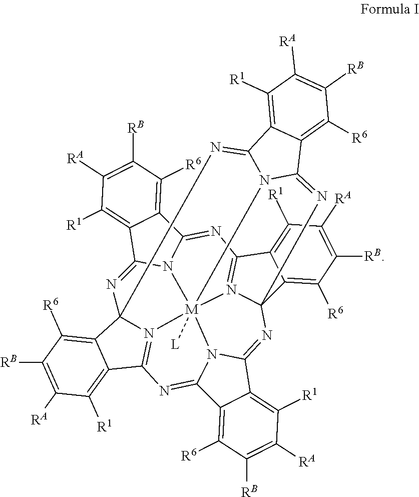

The variable M can be a metal. Herein metal atoms complexed with bridged phthalocyanine- and naphthalocyanine structures are drawn showing no valence state. However, the metal atoms have the appropriate valence state that is consistent with the structure shown (e.g., II, III, IV, V). The variable M can be a Group VIII or IX transition metal. The variable M can be Fe (e.g., Fe(III)). In some alternative embodiments, M can be Co. The axial ligand L can be a solvent molecule. The axial ligand L can be chosen from MeOH and H₂O. The axial ligand L can be H₂O.

In various aspects, the catalyst can be a purified catalyst. The catalyst can be purified by any suitable means. In various examples, the catalyst is purified according to techniques described in U.S. Pat. No. 10,065,980, which is incorporated by reference herewith in its entirety. In some aspects, the catalyst is purified via an embodiment of the method of purifying a catalyst described herein. In some aspects, the purified catalyst can exhibit certain properties not shown by the catalyst under impure conditions. For example, in some aspects, the purified catalyst can have exhibit different solubilities in various solvents, as compared to the catalyst in impure conditions. The purified catalyst can have any suitable purity, such as about 80 wt %—about 100 wt % pure, about 95 wt % to about 100 wt % pure, about 98 wt % to about 100 wt % pure, or about 80 wt % pure or less, or equal to or greater than about 81 wt %, 82, 83, 84, 85, 86, 87, 88, 89, 90, 91, 92, 93, 94, 95, 96, 97, 98, 99, 99.9, 99.99, or about 99.999 wt % pure or more.

At each occurrence, R^{A} and R^{B} can be independently chosen from —H, halide, an organic group, and a hydrophilic group, or R^{A} and R^{B} can together form a fused aromatic ring with the ring upon which R^{A} and R^{B} are substituted, R^{A} and R^{B} together having the structure:

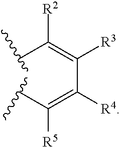

At each occurrence, R¹, R², R³, R⁴, R⁵, and R⁶ can be each independently chosen from —H, halide, an organic group, and a hydrophilic group. The hydrophilic group can be any suitable hydrophilic group. For example, at each occurrence, the hydrophilic group can be chosen from —C(O)OH, —O—C(O)OH, —P(O)(OH)₂, —OP(O)(OH)₂, —S(O)(O)OH, —OS(O)(O)OH, a salt thereof, a substituted or unsubstituted (C₁-C₅₀) hydrocarbyl ester thereof, and a combination thereof. The hydrophilic group can be —S(O)(O)OH.

In some aspects, R^{A} and R^{B} can have the structure:

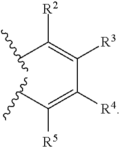

The variables R¹, R², R³, R⁴, R⁵, and R⁶ can be —H.

In some aspects, R^{A} and R^{B} can have the structure:

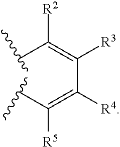

The variables R¹ and R⁶ can be —H. At each occurrence, R², R³, R⁴, and R⁵ can be independently chosen from —H and a hydrophilic group. At one more occurrences at least one of R², R³, R⁴, and R⁵ can be a hydrophilic group.

In some aspects, R^{A} and R^{B} can have the structure:

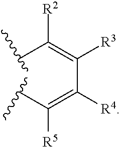

The variables R¹ and R⁶ can be —H. At each occurrence, R², R³, R⁴, and R⁵ can be independently chosen from —H and —S(O)(O)OH. At one more occurrences at least one of R², R³, R⁴, and R⁵ can be —S(O)(O)OH.

In some aspects, R¹, R^{A}, R^{B}, and R⁶ are —H. The catalyst can have the structure:

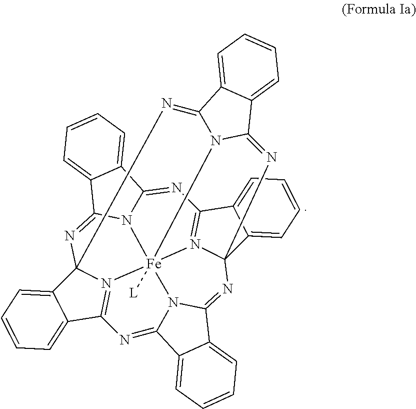

Axial ligand L can be H₂O.

The catalyst can have the structure:

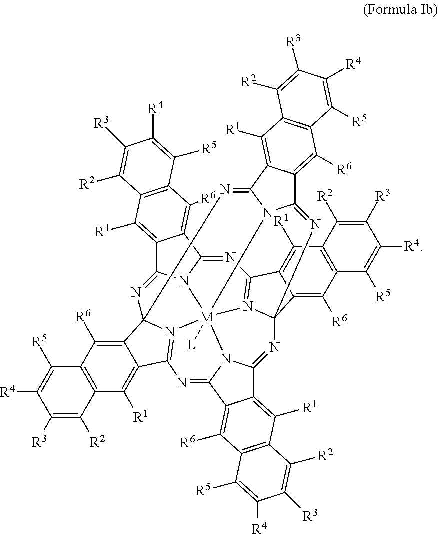

The variable M can be a metal. Herein metal atoms complexed with bridged phthalocyanine- and napththalocyanine structures are drawn showing no valence state. However, the metal atoms have the appropriate valence state that is consistent with the structure shown (e.g., II, III, IV, or V). The variable M can be a Group VIII or IX transition metal. The variable M can be Fe (e.g., Fe(III)). In some alternative embodiments, M can be Co. The axial ligand L can be a solvent molecule. The axial ligand L can be chosen from MeOH and H₂O. The axial ligand L can be H₂O.

At each occurrence, R¹, R², R³, R⁴, R⁵, and R⁶ can be independently chosen from —H, halide, an organic group, and a hydrophilic group. The hydrophilic group can be any suitable hydrophilic group. For example, at each occurrence, the hydrophilic group can be chosen from —C(O)OH, —O—C(O)OH, —P(O)(OH)₂, —OP(O)(OH)₂, —S(O)(O)OH, —OS(O)(O)OH, a salt thereof, a substituted or unsubstituted (C₁-C₅₀) hydrocarbyl ester thereof, and a combination thereof. The hydrophilic group can be —S(O)(O)OH.

In some aspects, R¹ and R⁶ are —H, and at each occurrence, R², R³, R⁴, and R⁵ are independently chosen from —H and a hydrophilic group. At one or more occurrences at least one of R², R³, R⁴, and R⁵ can be a hydrophilic group (e.g., at least one of R², R³, R⁴, or R⁵ in the molecule is a hydrophilic group).

In some aspects, R¹ and R⁶ are —H, and at each occurrence, R², R³, R⁴, and R⁵ are independently chosen from —H and —S(O)(O)OH. At one or more occurrences at least one of R², R³, R⁴, and R⁵ can be —S(O)(O)OH(e.g., at least one of R², R³, R⁴, or R⁵ in the molecule can be —S(O)(O)OH).

Various aspects of the present invention provide a method of oxidation including contacting an oxidizable starting material with the catalyst and an oxidant, to provide an oxidized product. The oxidizable starting material can be any suitable oxidizable starting material, such as a substituted or unsubstituted (C₁-C₅₀) hydrocarbyl alcohol, such as 2-pentanol, 1-pentanol, 2,4-dimethyl-3-pentanol, or isopropanol. The oxidant can be any suitable oxidant, such as tert-butylhydroperoxide, hydrogen peroxide, and combinations thereof. In various aspects, the contacting to provide an oxidized product can be carried out under solvent-containing or solvent-free conditions (e.g., wherein the reagents act as the solvent).

In various aspects, the present invention provides a method of forming the catalyst. For example, the method can include combining a suitable M-containing reagent (e.g., Fe(OAc)₂) with a suitable material, such as 2,3-naphthalenedicarbonitrile, under conditions sufficient to produce the catalyst.

In various aspects, the present invention provides a method of forming a derivatized catalyst. The method can include adding a hydrophilic group to the catalyst, such as by electrophilic aromatic substitution. The method can include adding to the catalyst one or more of —C(O)OH, —O—C(O)OH, —P(O)(OH)₂, —OP(O)(OH)₂, —S(O)(O)OH, —OS(O)(O)OH, a salt thereof, and a substituted or unsubstituted (C₁-C₅₀) hydrocarbyl ester thereof. The method can include adding to the catalyst-S(O)(O)OH, such as via treatment with fuming sulfuric acid.

In various aspects, the derivatized catalyst can have a greater water solubility than the un-derivatized catalyst, due to the added one or more hydrophilic groups. Various aspects of the present invention provide a method of oxidation including contacting a suitable oxidizable starting material, a suitable oxidant, the derivatized catalyst, and water.

### High Pressure Oxidation of Organic Substrates

The present disclosure provides a method of oxidizing an organic substrate, comprising contacting an organic substrate with an iron (III)-based phthalocyanine catalyst in a pressure vessel and providing molecular oxygen (O₂) at a pressure greater than atmospheric pressure to provide an oxidized product.

The molecular oxygen can be provided at a pressure can be 15 psi or greater, 150 psi or greater, or 1500 psi or greater. For example, the pressure can be about 100 psi to 1500 psi, 200 psi to 1000 psi, or 15 psi to 500 psi. In various aspects, the pressure is about, at least, or up to 15 psi, 20 psi, 25 psi, 30 psi, 35 psi, 40 psi, 45 psi, 50 psi, 55 psi, 60 psi, 65 psi, 70 psi, 75 psi, 80 psi, 85 psi, 90 psi, 95 psi, 100 psi, 110 psi, 120 psi, 130 psi, 140 psi, 150 psi, 160 psi, 170 psi, 180 psi, 190 psi, 200 psi, 210 psi, 220 psi, 230 psi, 240 psi, 250 psi, 260 psi, 270 psi, 280 psi, 290 psi, 300 psi, 310 psi, 320 psi, 330 psi, 340 psi, 350 psi, 360 psi, 370 psi, 380 psi, 390 psi, 400 psi, 410 psi, 420 psi, 430 psi, 440 psi, 450 psi, 460 psi, 470 psi, 480 psi, 490 psi, 500 psi, 550 psi, 600 psi, 650 psi, 700 psi, 750 psi, 800 psi, 850 psi, 900 psi, 950 psi, 1000 psi, 1050 psi, 1100 psi, 1150 psi, 1200 psi, 1250 psi, 1300 psi, 1350 psi, 1400 psi, 1450 psi, 1500 psi, 1550 psi, 1600 psi, 1650 psi, 1700 psi, 1750 psi, 1800 psi, 1850 psi, 1900 psi, 1950 psi, or 2000 psi. The reaction can be performed in a Parr high pressure variable temperature reactor with a glass liner.

In various aspects, the molecular oxygen utilized as oxidant is obtained via an air separator.

In various aspects, the reaction mixture of organic substrate, catalyst, and molecular oxygen is heated under pressure in the pressure vessel. For example, the method can involve heating the reaction mixture to a temperature of 25° C. to 250° C. The method can involve heating the reaction mixture to a temperature of 100° C. to 150° C. For example, the temperature can be about, at least, or up to 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., 160° C., 161° C., 162° C., 163° C., 164° C., 165° C., 166° C., 167° C., 168° C., 169° C., 170° C., 171° C., 172° C., 173° C., 174° C., 175° C., 176° C., 177° C., 178° C., 179° C., 180° C., 181° C., 182° C., 183° C., 184° C., 185° C., 186° C., 187° C., 188° C., 189° C., 190° C., 191° C., 192° C., 193° C., 194° C., 195° C., 196° C., 197° C., 198° C., 199° C., 200° C., 201° C., 202° C., 203° C., 204° C., 205° C., 206° C., 207° C., 208° C., 209° C., 210° C., 211° C., 212° C., 213° C., 214° C., 215° C., 216° C., 217° C., 218° C., 219° C., 220° C., 221° C., 222° C., 223° C., 224° C., 225° C., 226° C., 227° C., 228° C., 229° C., 230° C., 231° C., 232° C., 233° C., 234° C., 235° C., 236° C., 237° C., 238° C., 239° C., 240° C., 241° C., 242° C., 243° C., 244° C., 245° C., 246° C., 247° C., 248° C., 249° C., or 250° C. The method can involve heating the reaction mixture to a temperature of about or less than 150° C., 140° C., 130° C., 120° C., 110° C., 100° C., 90° C., 80° C., 70° C., 60° C., 50° C., 40° C., or 30° C.

In various aspects, the method can be performed as a solvent-free process. In other aspects, the method can be performed such that the reaction mixture of organic substrate, catalyst, and molecular oxygen is contacted together in a solvent in the pressure vessel. The solvent can be a multi-component solvent system. The solvent can be a biphasic solvent system. The method can be performed using a solvent that dissolves the catalyst. In other aspects, the method can be performed using a solvent that does not dissolve the catalyst. In various aspects, the method can be performed in the absence of solvent other than the oxidizable starting material.

In various aspects, the method can result in oxidation of the organic substrate and water. In various aspects where such oxidation approaches quantitative yields, the water can be the sole byproduct of oxidation of the organic substrate. In various aspects, the method can result in a product that is an epoxide, an alcohol, a ketone, an aldehyde, an acetal, a carboxylic acid, an ester, or a combination thereof.

The organic substrates used in the presently described method are not particularly limited. Indeed, a wide range of substrate types is demonstrated herein.

In various aspects, the organic substrate is a terminal alkene. In further aspects, when the organic substrate is a terminal alkene, the resulting product can be an aldehyde, a carboxylic acid, or a combination thereof.

In various aspects, the organic substrate is a internal alkene. In further aspects, when the organic substrate is an internal alkene, the resulting product can be an alcohol, a ketone, or a combination thereof.

In various aspects, the organic substrate is a primary alcohol. In further aspects, when the organic substrate is a primary alcohol, the resulting product can be an aldehyde, an acetal derived from condensation of the aldehyde and the primary alcohol, a carboxylic acid, an ester derived from condensation of the carboxylic acid and the primary alcohol, or a combination thereof.

In various aspects, the organic substrate is a secondary alcohol. In further aspects, when the organic substrate is a secondary alcohol, the resulting product can be a ketone, an acetal derived from condensation of the ketone and the primary alcohol, or a combination thereof.

In various aspects, the organic substrate is a styrene. In further aspects, when the organic substrate is a styrene, the resulting product can be benzaldehyde, benzoic acid, styrene oxide, oligomerization and/or polymerization products of styrene and/or of the oxidation products thereof, or a combination thereof.

In various aspects, the organic substrate is a toluene. In further aspects, when the organic substrate is a toluene, the resulting product can be benzaldehyde, benzoic acid, or a combination thereof.

The iron (III)-based phthalocyanine catalyst can be FeLX, FeLX8, or SFH(a sulfonated napthalocyanine analog of FeLX, see, for example, Formula Ic). The iron (III)-based phthalocyanine catalyst can have a structure according to Formula I. For example, the iron (III)-based phthalocyanine catalyst can be (L) (14,28-[1,3-diiminoisoindolinato]phthalocyaninato) Fe(III), where L represents an axially coordinated monodentate ligand consisting of a mixture of methanol and water in an approximately 4:1 ratio. The iron (III)-based phthalocyanine catalyst can also be (H₂O) (14,28-[1,3-diiminoisoindolinato]phthalocyaninato) Fe(III). The iron (III)-based phthalocyanine catalyst can be (H₂O) (18,36-[1,3-diimino-benzo[f]isoindole]phthalocyaninato) Fe(III). The iron (III)-based phthalocyanine catalyst can be a sulfonated iron (III)-based phthalocyanine catalyst. The iron (III)-based phthalocyanine catalyst can be sulfonated (H₂O) (18,36-[1,3-diimino-benzo[f]isoindole]phthalocyaninato) Fe(III). The iron (III)-based phthalocyanine catalyst can have the general formula [C₆₀H₍₃₀₋ₓ₎FeN₁₁O₁₅S₅]^{x-}, where X represents number of sulfonate group, and wherein the charge compensating cations are sodium ions. In various aspects, the iron (III)-based phthalocyanine catalyst is alcohol soluble. In various aspects, the iron (III)-based phthalocyanine catalyst is water soluble. The iron (III)-based phthalocyanine catalyst can be configured as a homogeneous catalyst. The iron (III)-based phthalocyanine catalyst can be configured as a heterogenous catalyst. The iron (III)-based phthalocyanine catalyst can be a heterogenous catalyst supported on charcoal or ion exchange resins.

In various other aspects, the presently described oxidation method can utilize a catalyst can having the structure:

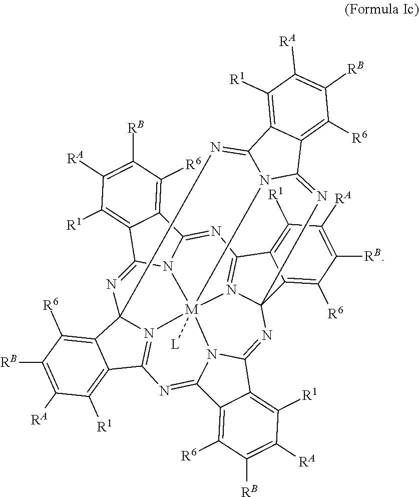

M can be a metal; the axial ligand L can be a solvent molecule; at each occurrence, R^{A} and R^{B} can be independently chosen from —H, halide, an organic group, and a hydrophilic group, or R^{A} and R^{B} can together form a fused aromatic ring with the ring upon which R^{A} and R^{B} are substituted; and R^{A} and R^{B} together can have the structure:

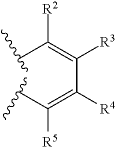

At each occurrence, R¹, R², R³, R⁴, R⁵, and R⁶ can be each independently chosen from —H, halide, an organic group, and a hydrophilic group. In various further aspects, each occurrence, the hydrophilic group is chosen from —C(O)OH, —O—C(O)OH, —P(O)(OH)₂, —OP(O)(OH)₂, —S(O)(O)OH, —OS(O)(O)OH, a salt thereof, a substituted or unsubstituted (C₁-C₅₀) hydrocarbyl ester thereof, and a combination thereof. In yet further aspects, the hydrophilic group is the hydrophilic group is —S(O)(O)OH. In further examples, at one more occurrences at least one of R², R³, R⁴, and R⁵ is —S(O)(O)OH. In a preferred aspect, M is Fe. L can be CH₃CN, MeOH, H₂O, or a combination thereof. For example, L can be a mixture of MeOH and H₂O in any ratio from 100:1 to 1:100.

In various aspects, the catalyst can have the structure:

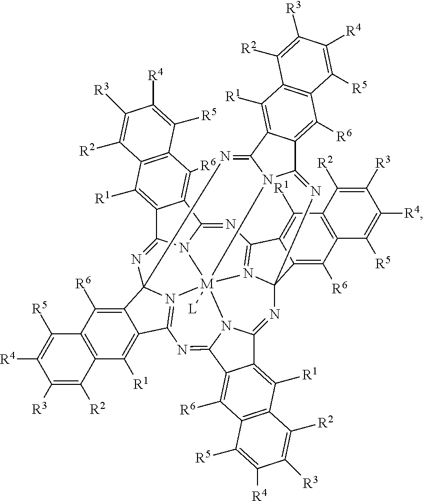

M can be a metal; the axial ligand L can be a solvent molecule; at each occurrence, R^{A} and R^{B} can be independently chosen from —H, halide, an organic group, and a hydrophilic group. In a preferred aspect, M is Fe. L can be CH₃CN, MeOH, H₂O, or a combination thereof. For example, L can be a mixture of MeOH and H₂O in any ratio from 100:1 to 1:100.

The method can include contacting an oxidizable starting material with any embodiment of a catalyst described herein and molecular oxygen. The contacting of the oxidizable starting material, the catalyst, and the molecular oxygen, provides an oxidized product. In some embodiments, the catalyst is an embodiment of the catalyst described herein. The catalyst can be unpurified or purified, such as having any suitable purity, such as about 80 wt % to about 100 wt % pure, about 95 wt % to about 100 wt % pure, about 98 wt % to about 100 wt % pure, or about 80 wt % pure or less, or equal to or greater than about 81 wt %, 82, 83, 84, 85, 86, 87, 88, 89, 90, 91, 92, 93, 94, 95, 96, 97, 98, 99, 99.9, 99.99, or about 99.999 wt % pure or more.

In various aspects, the contacting to provide an oxidized product can be carried out under solvent-free, i.e., neat, conditions. Under some such solvent-free conditions, the liquid reagents (e.g., the oxidizable starting material and the oxidized product) can be suitable for dissolving the catalyst and the oxidizing agent. For example, in various embodiments, the catalyst can be soluble in non-aromatic alcohols, such that oxidation of such non-aromatic alcohols can be carried out without the addition of any other solvents. In some embodiments, the catalyst remains undissolved and operates catalytically from a heterogeneous solution. The catalyst can be an unsupported catalyst, or the catalyst can comprise a solid support, such as carbon (e.g., activated charcoal, activated carbon, or graphene), or such as any suitable solid support (e.g., alumina or silica). In various aspects, the support can be an ion exchange resin. In various aspects, the support can be a crosslinked polystyrene resin. In various aspects, the support can be Amberlite resin.

The oxidizable starting material can be any suitable oxidizable starting material. The oxidizable starting material can be alkanes, alkenes, alcohols, styrenes, toluenes, any of which linear or cyclic, and any of which can be substituted or unsubstituted. Further, the oxidizable starting material can be a substrate that contains cycloalkane (e.g., a cyclohexane moiety), a primary alcohol (e.g., an 1-butanol moiety), a secondary alcohol (e.g., 2-butanol moiety), a styrene moiety, an acyclic alkene (e.g., a 3-hexene moiety), a cyclic alkene (e.g., a cyclohexene moiety), or a benzylic sp3-hybridized carbon group (e.g., a toluene moiety).

In various aspects, the oxidizable organic substrate starting material can be a substituted or unsubstituted (C₁-C₅₀) hydrocarbyl alcohol. In various aspects, the oxidizable starting material can be a substituted or unsubstituted (C₃-C₅₀)alkene. In various aspects, the oxidizable starting material can be a substituted or unsubstituted (C₅-C₅₀)cycloalkene. The alkene can be a terminal alkene group or a non-terminal alkene group. The alkene can be a trans or cis alkene. The oxidizable starting material can be styrene, for example styrene or stilbene.

In various aspects, the oxidizable organic substrate starting material can be an alcohol readily obtained from renewable sources such as agricultural waste. The alcohol can be a bioalcohol. Use of bioalcohols and waste alcohols has the advantages of environmental sustainability particularly when adopted for commodity chemical production on a commercial/industrial scale.

In various aspects, the reaction conditions can result in excellent efficiency. For example, the reaction conditions can achieve a turnover number (e.g., the moles of product produced divided by the moles of catalyst used) of about 200 to about 20,000, about 500 to about 15,000, about 300 to about 1,000, or about 200 or less, or about 250, 300, 350, 400, 450, 500, 550, 600, 650, 700, 750, 800, 850, 900, 950, 1,000, 1,100, 1,200, 1,300, 1,400, 1,500, 1,600, 1,800, 2,000, 2,500, 3,000, 4,000, 5,000, 6,000, 8,000, or about 10,000 or more. During the contacting to provide the oxidized product, the catalyst can have any suitable turnover frequency (e.g., turnover number divided by reaction time). In further aspects, the turnover frequency can be about 0.001 s⁻¹ to about 1 s⁻¹, about 0.01 s⁻¹ to about 0.5 s⁻¹, about 0.01 s⁻¹ to about 0.15 s⁻¹, or about or faster than 0.01 s⁻¹, 0.02 s⁻¹, 0.03 s⁻¹, 0.04 s⁻¹, 0.05 s⁻¹, 0.06 s⁻¹, 0.07 s⁻¹, 0.08 s⁻¹, 0.09 s⁻¹, 0.1 s⁻¹, 0.11 s⁻¹, 0.12 s⁻¹, 0.13 s⁻¹, 0.14 s⁻¹, 0.15 s⁻¹, 0.160 s⁻¹, 0.17 s⁻¹, 0.18 s⁻¹, 0.19 s⁻¹, 0.2 s⁻¹, 0.25 s⁻¹, 0.3 s⁻¹, 0.35 s⁻¹, 0.4 s⁻¹, 0.45 s⁻¹, 0.5 s⁻¹, 0.55 s⁻¹, 0.6 s⁻¹, 0.65 s⁻¹, 0.7 s⁻¹, 0.75 s⁻¹, or 0.8 s⁻¹. In some aspects, the turnover frequency can be about 500 h⁻¹ to about 20,000 h⁻¹, about 1,000 h⁻¹ to about 4,000 h⁻¹, about 500 h⁻¹ or less, or about 600 h⁻¹, 800, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 4,000, 4,500, 5,000, 6,000, 7,000, 8,000, 9,000, 10,000, 15,000, or about 20,000 h⁻¹ or more.

### EXAMPLES

Various aspects of the present disclosure can be better understood by reference to the following Examples which are offered by way of illustration. The invention is not limited to the Examples given herein.

### Materials and Methods

Catalysts. All catalysts were synthesized from commercially available starting materials according to the methods described in U.S. Pat. Nos. 10,065,980; 11,826,740, each of which are incorporated by reference herewith in their entirety.

Molecular Oxygen Oxidation. All reactions were performed in a pressure vessel equipped with a feed of molecular oxygen (O₂). Suitable pressure vessels are available from the Parr Instrument Company (Moline, IL). For example, a Series 4560 Mini Reactor, Series 4520 Bench Top Reactor, or Series 4555 General Purpose Floor Stand Reactor, as suitable based on volume of the reaction mixture. Molecular oxygen can be prepared by use of an air separator, which are available from Fisher Scientific (Waltham, MA). Across all reactions, molecular oxygen was provided at greater than atmospheric pressure.

### Oxidation of Secondary Alcohols

Oxidation of isobutyl alcohol to 2-butanone. The pressure vessel was charged with the catalyst and isobutyl alcohol neat without any added solvent. At a first set of conditions, treatment with molecular oxygen at 100° C. and elevated pressure resulted in 2-butanone as the sole product. At a second set of conditions, treatment with molecular oxygen at 140° C. resulted in nearly 100% conversion to 2-butanone, but with a minor byproduct of ethyl acetate, reflecting further oxidation of the primary product. The turnover number (TON) for methyl ethyl ketone alone is 9600. Adding ethyl acetate to give total oxidized product, TON is 11,000 with a corresponding turnover frequency (TOF) of 8.7 min-1 (or 0.145 s⁻¹), within the realm of TOF values considered viable in industrial/commercial synthesis.

Oxidation of 2-pentanol to 2-pentanone. The pressure vessel was charged with the catalyst and 2-pentanol neat without any added solvent. Various conditions were tested, each involving treatment with molecular oxygen at elevated pressure. Very good selectivity of 2-pentanone was achieved. Treatment with molecular oxygen at 140° C. resulted in 2-pentanone as the primary product with a turnover number (TON) of 7600, with a turnover frequency (TOF) of 0.11 s⁻¹.

Oxidation of 2-propanol to acetone. The pressure vessel was charged with the catalyst and 2-propanol neat without any added solvent. Excellent selectivity of acetone was achieved. Treatment with molecular oxygen at 150° C. resulted in acetone with a turnover number (TON) of 7,000, with a turnover frequency (TOF) of 0.81 s⁻¹.

### Oxidation of Primary Alcohols

Oxidation of ethanol to acetic acid. The pressure vessel was charged with the catalyst and ethanol neat without any added solvent. Various conditions were tested, each involving treatment with molecular oxygen at elevated pressure. Acetic acid was the major product, with a secondary product of ethyl acetate, reflecting condensation of the primary product with starting material. A very minor product of 1,1-diethoxyethane, an acetal of acetaldehyde was also present. Under exemplary conditions, a turnover number (TON) of 13,000 and a corresponding turnover frequency (TOF) of 0.18 s⁻¹ taking into account all oxidized products was achieved.

Oxidation of 1-proponol to propanal. The pressure vessel was charged with the catalyst and 1-proponol neat without any added solvent. Various conditions were tested, each involving treatment with molecular oxygen at elevated pressure. Good selectivity was achieved with the primary product being 1,1-dipropoxypropane, which is the result of acetal formation of the resulting propanal with starting material. This reaction was more sensitive to reaction conditions, particularly temperature, relative to the example utilizing ethanol as a substrate.

Oxidation of methanol to methylal. The pressure vessel was charged with the catalyst and methanol neat without any added solvent. Various conditions were tested, each involving treatment with molecular oxygen at elevated pressure. Good selectivity was achieved with the primary product being methylal, which is the result of acetal formation of the resulting formaldehyde with starting material. Methyl formate also formed as a second product, which is the result of condensation between the methanol starting material and formic acid that resulted from oxidation of methylal. Under exemplary conditions, taking into account both oxidation products, a turnover number (TON) of 2,200 and a turnover frequency (TOF) of 0.025 s⁻¹ was achieved.

### Oxidation of Acyclic Olefins.

Oxidation of trans-3-hexene to various oxidation products. The pressure vessel was charged with the catalyst and trans-3-hexene. Various conditions were tested each involving treatment with molecular oxygen at elevated pressure. Homogenous reaction conditions employing FeLX in a mixed dichloromethane/acetonitrile solvent system were examined. Oxidation provided access to the epoxide of trans-3-hexene, the alpha, beta-unsaturated ketone of trans-3-hexene, and the alpha, beta-unsaturated alcohol of trans-3-hexene. Lower O₂ pressures resulted in greater proportions of epoxide product.

Oxidation of trans-stilbene to various oxidation products. The pressure vessel was charged with the catalyst and trans-stilbene. Various conditions were tested each involving treatment with molecular oxygen at elevated pressure. Oxidation provided benzaldehyde, benzophenone, and stilbene oxide. Various solvent systems were explored, in each case providing either benzophenone or benzaldehyde as the major product.

### Oxidation of Cyclic Olefins

Oxidation of cyclohexene to various oxidation products. The pressure vessel was charged with the catalyst and cyclohexene. Close to 100% conversion to oxidized products was obtained. Various conditions were tested, including both homogeneous and heterogenous systems, each involving treatment with molecular oxygen at elevated pressure. Lower O₂ pressures resulted in cyclohexene oxide formation, but not as a sole product. Higher O₂ pressures favored formation of 2-cyclohexene-1-one as the major product. Under various conditions, 2-cyclohexene-1-ol was also formed. Biphasic reactions utilizing SFH, a water-soluble catalyst type, gave very good selectivity for 2-cyclohexene-1-one. Heterogenous systems where the catalyst (either FeLX or SFH) was immobilized on a solid support (charcoal or Amberlite resin, respectively) gave 2-cyclohexene-1-one as the major product. Catalysts on solid support were recyclable.

Oxidation of cyclopentene to various oxidation products. The pressure vessel was charged with the catalyst and cyclopentene. Close to 100% conversion to oxidized products was obtained. Various conditions were tested, each involving treatment with molecular oxygen at elevated pressure. Conditions could be adjusted to obtain cyclopentene oxide as a major product, but not as a sole product. Very clean production of 2-cyclopentene-1-one was achieved utilizing neat conditions, where no solvent other than cyclopentene was utilized for the reaction system.

Oxidation of norbornene to various oxidation products. The pressure vessel was charged with the catalyst and norbornene. Close to 100% conversion to oxidized products was obtained. Various conditions were tested, each involving treatment with molecular oxygen at elevated pressure. Multiple oxidation products can be accessed, but can be complicated due to potential rearrangement reactions of various oxidation products of this bicyclic system.

Oxidation of (+)-alpha-pinene to various oxidation products. The pressure vessel was charged with the catalyst and (+)-alpha-pinene. Various conditions were tested, each involving treatment with molecular oxygen at elevated pressure. The primary products achieved were the alpha, beta-unsaturated ketone of (+)-alpha-pinene and the epoxide of (+)-alpha-pinene.

### Oxidation of Styrene Compounds

Oxidation of styrene to benzaldehyde. The pressure vessel was charged with the catalyst and styrene. Various conditions were tested, each involving treatment with molecular oxygen at elevated pressure. Higher temperature conditions resulted in polymerization products, but use of lower temperatures was effective to prevent polymerization. Under exemplary conditions, benzaldehyde formation was achieved.

Oxidation of trans-beta-methylstyrene to benzaldehyde. The pressure vessel was charged with the catalyst and trans-beta-methylstyrene. Various conditions were tested, each involving treatment with molecular oxygen at elevated pressure. The primary product was benzaldehyde, but small amounts of epoxidated trans-beta-methylstyrene were observed. Lower temperatures appeared to provide greater amounts of the epoxide. Use of biphasic system with aqueous-soluble catalyst resulted in some benzoic acid formation.

### Oxidation of Cycloalkanes.

Oxidation of cyclohexane to various oxidation products. The pressure vessel was charged with the catalyst and cyclohexane. Various conditions were tested, each involving treatment with molecular oxygen at elevated pressure. At a first set of conditions utilizing heterogenous FeLX immobilized on charcoal, treatment with molecular oxygen at 100° C. and elevated pressure resulted in both cyclohexanol and cyclohexanone, but conversion remained low. Using these same reaction conditions but raising the temperature to 130° C. resulted in a complex mixture.

Oxidation of toluene to benzaldehyde. The pressure vessel was charged with the catalyst and toluene neat without any added solvent. At a first set of conditions, treatment with molecular oxygen at 110° C. and elevated pressure resulted in benzaldehyde as the major product, but low conversion, with a trace amount of benzoic acid observed. Using these same reaction conditions but raising the temperature to 130° C. resulted in a complex mixture.

### SUMMARY

The yield and selectivity of oxidation products was controllable by adjusting temperature, reaction time, presence or absence of solvent, solvent system selection, substrate concentration, catalyst concentration, and pressure of molecular oxygen.

### Additional Embodiments

The following exemplary embodiments are provided, the numbering of which is not to be construed as designating levels of importance:
Embodiment 1 provides a method of oxidizing an organic substrate, comprising contacting an organic substrate with an iron (III)-based phthalocyanine catalyst in a pressure vessel and providing O₂ at a pressure greater than atmospheric pressure to provide an oxidized product.
Embodiment 2 provides the method of Embodiment 1, wherein the pressure is 15 psi or greater.
Embodiment 3 provides the method of any one of Embodiments 1 to 2, wherein the pressure is 150 psi or greater.
Embodiment 4 provides the method of any one of Embodiments 1 to 3, wherein the pressure is 150 psi to 1500 psi.
Embodiment 5 provides the method of any one of Embodiments 1 to 4, is performed in a Parr high pressure variable temperature reactors with a glass liner.
Embodiment 6 provides the method of any one of Embodiments 1 to 5, wherein the catalyst is (L) (14,28-[1,3-diiminoisoindolinato]phthalocyaninato) Fe(III), where L represents an axially coordinated monodentate ligand consisting of a mixture of methanol and water in an approximately 4:1 ratio.
Embodiment 7 provides the method of any one of Embodiments 1 to 6, wherein the catalyst is (H₂O) (14,28-[1,3-diiminoisoindolinato]phthalocyaninato) Fe(III).
Embodiment 8 provides the method of any one of Embodiments 1 to 7, wherein the catalyst is (H₂O) (18,36-[1,3-diimino-benzo[f]isoindole]phthalocyaninato) Fe(III).
Embodiment 9 provides the method of any one of Embodiments 1 to 8, wherein the catalyst is sulfonated iron (III)-based phthalocyanine catalyst.
Embodiment 10 provides the method of any one of Embodiments 1 to 9, wherein the catalyst is sulfonated (H₂O) (18,36-[1,3-diimino-benzo[f]isoindole]phthalocyaninato) Fe(III).
Embodiment 11 provides the method of any one of Embodiments 1 to 10, wherein the catalyst has the general formula [C₆₀H₍₃₀₋ₓ₎FeN₁₁O₁₅S₅]^{x-}, where X represents number of sulfonate group, and wherein the charge compensating cations are sodium ions.
Embodiment 12 provides the method of any one of Embodiments 1 to 11, wherein the catalyst is alcohol soluble.
Embodiment 13 provides the method of any one of Embodiments 1 to 12, wherein the catalyst is water soluble.
Embodiment 14 provides the method of any one of Embodiments 1 to 13, wherein the catalyst is a homogeneous catalyst.
Embodiment 15 provides the method of any one of Embodiments 1 to 14, wherein the catalyst is a heterogenous catalyst supported on charcoal or ion exchange resins.
Embodiment 16 provides the method of any one of Embodiments 1 to 15, wherein the O₂ is obtained via an air separator.
Embodiment 17 provides the method of any one of Embodiments 1 to 16, wherein the temperature is 25° C. to 250° C.
Embodiment 18 provides the method of any one of Embodiments 1 to 17, wherein the temperature is 100° C. to 150° C.
Embodiment 19 provides the method of any one of Embodiments 1 to 18, wherein the temperature is 140° C. or less.
Embodiment 20 provides the method of any one of Embodiments 1 to 19, which is performed as a solvent-free process.
Embodiment 21 provides the method of any one of Embodiments 1 to 20, which is performed with a solvent.
Embodiment 22 provides the method of any one of Embodiments 1 to 21, which is performed with a biphasic solvent system.
Embodiment 23 provides the method of any one of Embodiments 1 to 22, which is performed with a solvent that dissolves the catalyst.
Embodiment 24 provides the method of any one of Embodiments 1 to 23, which is performed with a solvent that does not dissolve the catalyst.
Embodiment 25 provides the method of any one of Embodiments 1 to 24, wherein the sole byproduct of oxidation of the organic substrate is water.
Embodiment 26 provides the method of any one of Embodiments 1 to 25, wherein the contacting is performed in the absence of solvent other than the oxidizable starting material.
Embodiment 27 provides the method of any one of Embodiments 1 to 26, wherein the product is an epoxide, an alcohol, a ketone, an aldehyde, an acetal, a carboxylic acid, an ester, or a combination thereof.
Embodiment 28 provides the method of any one of Embodiments 1 to 27, wherein the organic substrate is a terminal alkene, and wherein the oxidized group is an aldehyde, a carboxylic acid, or a combination thereof.
Embodiment 29 provides the method of any one of Embodiments 1 to 28, wherein the organic substrate is an internal alkene, and wherein the oxidized group is an alcohol, a ketone, or a combination thereof
Embodiment 30 provides the method of any one of Embodiments 1 to 29, wherein the organic substrate is a primary alcohol, and wherein the oxidized group is an aldehyde, an acetal derived from condensation of the aldehyde and the primary alcohol, a carboxylic acid, an ester derived from condensation of the carboxylic acid and the primary alcohol, or a combination thereof.
Embodiment 31 provides the method of any one of Embodiments 1 to 30, wherein the organic substrate is a secondary alcohol, and wherein the oxidized group is a ketone, an acetal derived from condensation of the ketone and the primary alcohol, or a combination thereof.
Embodiment 32 provides the method of any one of Embodiments 1 to 31, wherein the organic substrate is a styrene and the oxidized product is benzaldehyde, benzoic acid styrene oxide, oligomerization and/or polymerization products of styrene and/or of the oxidation products thereof, or a combination thereof.
Embodiment 33 provides the method of any one of Embodiments 1 to 32, wherein the catalyst has the structure:

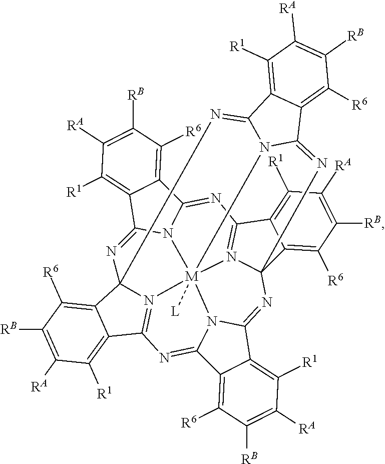

wherein
M is a metal,
axial ligand L is a solvent molecule,
at each occurrence, R^{A} and R^{B} are independently chosen from —H, halide, an organic group, and a hydrophilic group, or R^{A} and R^{B} together form a fused aromatic ring with the ring upon which R^{A} and R^{B} are substituted, R^{A} and R^{B} together having the structure:

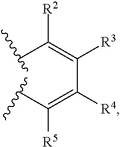

and
at each occurrence, R¹, R², R³, R⁴, R⁵, and R⁶ are each independently chosen from —H, halide, an organic group, and a hydrophilic group.
Embodiment 34 provides the method of any one of Embodiments 1 to 33, wherein at each occurrence, the hydrophilic group is chosen from —C(O)OH, —O—C(O)OH, —P(O)(OH)₂, —OP(O)(OH)₂, —S(O)(O)OH, —OS(O)(O)OH, a salt thereof, a substituted or unsubstituted (C₁-C₅₀) hydrocarbyl ester thereof, and a combination thereof.
Embodiment 35 provides the method of any one of Embodiments 1 to 34, wherein the hydrophilic group is the hydrophilic group is —S(O)(O)OH.
Embodiment 36 provides the method of any one of Embodiments 1 to 35, wherein at one more occurrences at least one of R², R³, R⁴, and R⁵ is —S(O)(O)OH.
Embodiment 37 provides the method of any one of Embodiments 1 to 36, wherein M is Fe.
Embodiment 38 provides the method of any one of Embodiments 1 to 37, wherein the catalyst has the structure:

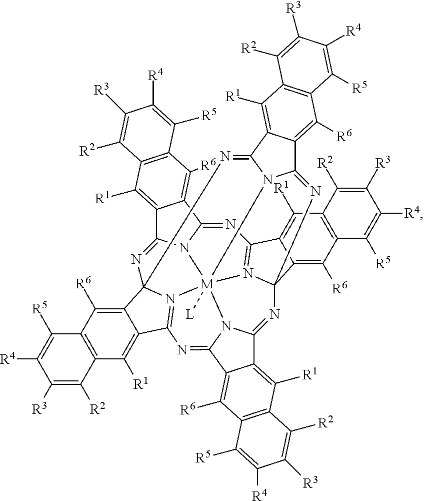

wherein
   M is a metal,
   L is a solvent molecule, and

at each occurrence, R¹, R², R³, R⁴, R⁵, and R⁶ are independently chosen from —H, halide, an organic group, and a hydrophilic group.
Embodiment 39 provides the method of any one of Embodiments 1 to 38, wherein L is CH₃CN, MeOH, H₂O, or a combination thereof.
Embodiment 40 provides the method of any one of Embodiments 1 to 39, wherein L is H₂O.
Embodiment 41 provides the method of any one or any combination of Embodiments 1-40 optionally configured such that all elements or options recited are available to use or select from.

What is claimed is:

1. A method of oxidizing an organic substrate, comprising contacting an organic substrate with an iron (III)-based phthalocyanine catalyst in a pressure vessel and providing O₂ at a pressure greater than atmospheric pressure to provide an oxidized product.

2. The method of claim 1, wherein the pressure is 15 psi or greater.

3. The method of claim 1, wherein the pressure is 150 psi or greater.

4. The method of claim 1, wherein the pressure is 150 psi to 1500 psi.

5. The method of claim 1, is performed in a Parr high pressure variable temperature reactor with a glass liner.

6. The method of claim 1, wherein the O₂ is obtained via an air separator.

7. The method of claim 1, wherein the temperature is 25° C. to 250° C.

8. The method of claim 1, wherein the temperature is 140° C. or less.

9. The method of claim 1, which is performed as a solvent-free process.

10. The method of claim 1, which is performed with a solvent.

11. The method of claim 1, which is performed with a biphasic solvent system.

12. The method of claim 1, wherein the organic substrate is a bioalcohol.

13. The method of claim 1, wherein the organic substrate is derived from an agricultural waste product.

14. The method of claim 1, wherein the organic substrate is a terminal alkene, and wherein the oxidized group is an aldehyde, a carboxylic acid, or a combination thereof.

15. The method of claim 1, wherein the organic substrate is an internal alkene, and wherein the oxidized group is an alcohol, a ketone, or a combination thereof.

16. The method of claim 1, wherein the organic substrate is a primary alcohol, and wherein the oxidized group is an aldehyde, an acetal derived from condensation of the aldehyde and the primary alcohol, a carboxylic acid, an ester derived from condensation of the carboxylic acid and the primary alcohol, or a combination thereof.

17. The method of claim 1, wherein the organic substrate is a secondary alcohol, and wherein the oxidized group is a ketone, an acetal derived from condensation of the ketone and the primary alcohol, or a combination thereof.

18. The method of claim 1, wherein the organic substrate is a styrene and the oxidized product is benzaldehyde, benzoic acid, styrene oxide, oligomerization and/or polymerization products of styrene and/or of the oxidation products thereof, or a combination thereof.

19. The method of claim 1, which results in a turnover frequency for the catalyst of 0.145 s⁻¹ or faster.

20. The method of claim 1, wherein the catalyst has the structure: 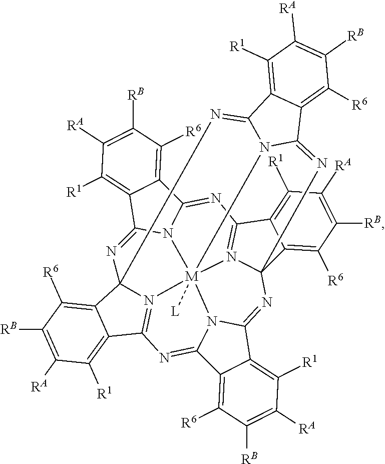
wherein
M is a metal, axial ligand L is a solvent molecule, at each occurrence, R^{A} and R^{B} are independently chosen from —H, halide, an organic group, and a hydrophilic group, or R^{A} and R^{B} together form a fused aromatic ring with the ring upon which R^{A} and R^{B} are substituted, R^{A} and R^{B} together having the structure: 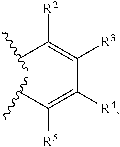
and
at each occurrence, R¹, R², R³, R⁴, R⁵, and R⁶ are each independently chosen from —H, halide, an organic group, and a hydrophilic group.
